(19) United States
(12) Patent Application Publication
(10) Pub. No.: US 2023/0277772 A1
(43) Pub. Date: Sep. 7, 2023

(54) NEEDLE-FREE INJECTOR

(71) Applicant: JEISYS MEDICAL INC., Seoul (KR)

(72) Inventors: Myeong Wook GU, Seoul (KR); Yeong Gi JEON, Seoul (KR); Kyu Young SEO, Seoul (KR); Seung Wook LEE, Seoul (KR); Si Youn KIM, Seoul (KR); Won Ju YI, Seoul (KR); Dong Hwan KANG, Seoul (KR)

(73) Assignee: JEISYS MEDICAL INC., Seoul (KR)

(21) Appl. No.: 18/315,593

(22) Filed: May 11, 2023

Related U.S. Application Data
(63) Continuation of application No. PCT/KR2021/014706, filed on Oct. 20, 2021.

(30) Foreign Application Priority Data
Dec. 15, 2020 (KR) 10-2020-0175289

(51) Int. Cl. A61M 5/30; A61M 5/315

(52) U.S. Cl. CPC A61M 5/30; A61M 5/315; A61M 2205/8287

(57) ABSTRACT

Disclosed is a needle-free injector. The needle-free injector includes a first current applying unit that applies a first current, a first coil that receives the first current from the first current applying unit, a second coil that moves to apply pressure to a drug, a housing of which the outside is surrounded by the first coil and in which the second coil is positioned, a drug chamber which is connected to the housing and in which the drug is positioned, and an injection nozzle positioned in the drug chamber to inject the drug.

### CROSS-REFERENCE TO RELATED APPLICATIONS

The present application is a continuation of International Patent Application No. PCT/KR2021/014706, filed on Oct. 20, 2021, which is based upon and claims the benefit of priority to Korean Patent Application No. 10-2020-0175289 filed on Dec. 15, 2020. The disclosures of the above-listed applications are hereby incorporated by reference herein in their entirety.

### BACKGROUND

Embodiments of the inventive concept described herein relate to a needle-free injector using a magnetic field.

A drug delivery system is designed to efficiently deliver the required amount of drug into a body such that side effects occurring in an existing method are minimized and therapeutic effects of medicines are maximized, when medicines for treating diseases or wounds in a human body are used.

An injection method most commonly used in the drug delivery system may inject drugs accurately and efficiently. However, the injection method has several disadvantages, such as injection phobia due to pain during injection, risk of infection due to reuse, and a large amount of medical waste.

To solve these issues, drug delivery methods such as needle-free injectors are being developed.

For example, a liquid injection technology that is one of the needle-free injector technologies is a technology that thermally expands liquid by applying shock waves through laser or electric waves to the liquid and generates a high-speed liquid stream by using the pressure generated during the thermal expansion so as to inject the liquid into a skin.

However, the conventional liquid injection technology has a complicated structure and is difficult to be miniaturized.

### SUMMARY

The present disclosure has been made to solve the above-mentioned problems occurring in the prior art, an object of the present disclosure is to present disclosure provide a needle-free injector using a magnetic field, which has a simple structure and is easy to be miniaturized by using a magnetic field.

Another object of the present disclosure is to present disclosure provide a needle-free injector using a magnetic field, which applies current, moves the coil, injects a drug, and restores the original form.

According to an embodiment, a needle-free injector using a magnetic field includes a first current applying unit that applies a first current, a first coil that receives the first current from the first current applying unit, a second coil that moves to apply pressure to a drug, a housing of which the outside is surrounded by the first coil and in which the second coil is positioned, a drug chamber which is connected to the housing and in which the drug is positioned, and an injection nozzle positioned in the drug chamber to inject the drug.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1A is a cross-sectional view schematically illustrating a situation before voltage is applied to a needle-free injector by using a magnetic field, according to an embodiment of the present disclosure;

FIG. 1B is a cross-sectional view schematically illustrating that a drug is injected by applying voltage to a needle-free injector by using a magnetic field, according to an embodiment of the present disclosure;

FIG. 2A is a cross-sectional view schematically illustrating a situation before voltage is applied to a needle-free injector by using a magnetic field, according to an embodiment of the present disclosure;

FIG. 2B is a cross-sectional view schematically illustrating that a drug is injected by applying voltage to a needle-free injector by using a magnetic field, according to an embodiment of the present disclosure;

FIG. 3A is a cross-sectional view schematically illustrating a situation before voltage is applied to a needle-free injector by using a magnetic field, according to an embodiment of the present disclosure;

FIG. 3B is a cross-sectional view schematically illustrating that a drug is injected by applying voltage to a needle-free injector by using a magnetic field, according to an embodiment of the present disclosure; and

FIG. 4 shows a current of each of a first coil and a second coil over time when a pulse current of one cycle is applied to a needle-free injector using a magnetic field shown in FIGS. 1A and 1B, according to an embodiment of the present disclosure.

### DETAILED DESCRIPTION

The above and other aspects, features and advantages of the present disclosure will become apparent from embodiments to be described in detail in conjunction with the accompanying drawings. The present disclosure, however, may be embodied in various different forms, and should not be construed as being limited only to the illustrated embodiments. Rather, these embodiments are provided as examples so that the present disclosure will be thorough and complete, and will fully convey the scope of the present disclosure to those skilled in the art. The present disclosure may be defined by the scope of the claims.

The terms used herein are provided to describe embodiments, not intended to limit the present disclosure. In the specification, the singular forms include plural forms unless particularly mentioned. The terms “comprises” and/or “comprising” used herein do not exclude the presence or addition of one or more other components, in addition to the aforementioned components. The same reference numerals denote the same components throughout the specification. As used herein, the term “and/or” includes each of the associated components and all combinations of one or more of the associated components. It will be understood that, although the terms “first”, “second”, etc., may be used herein to describe various components, these components should not be limited by these terms. These terms are only used to distinguish one component from another component. Thus, a first component that is discussed below could be termed a second component without departing from the technical idea of the present disclosure.

Unless otherwise defined, all terms (including technical and scientific terms) used herein have the same meaning as commonly understood by those skilled in the art to which the present disclosure pertains. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein.

Hereinafter, embodiments of the present disclosure will be described in detail with reference to accompanying drawings.

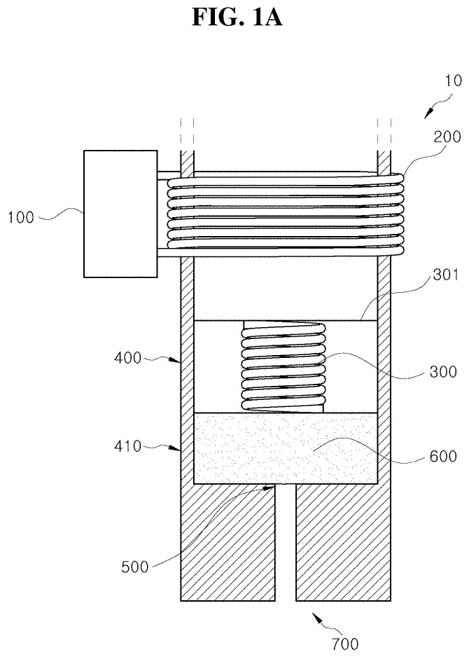

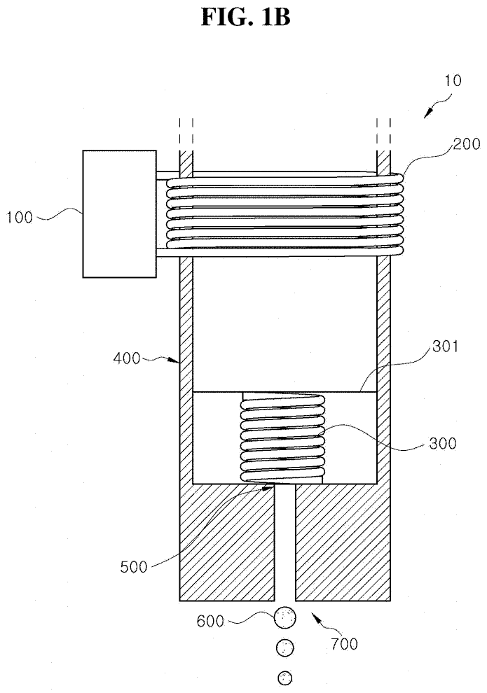

FIG. 1A is a cross-sectional view schematically illustrating a situation before voltage is applied to a needle-free injector by using a magnetic field, according to an embodiment of the present disclosure. FIG. 1B is a cross-sectional view schematically illustrating that a drug is injected by applying voltage to a needle-free injector by using a magnetic field, according to an embodiment of the present disclosure.

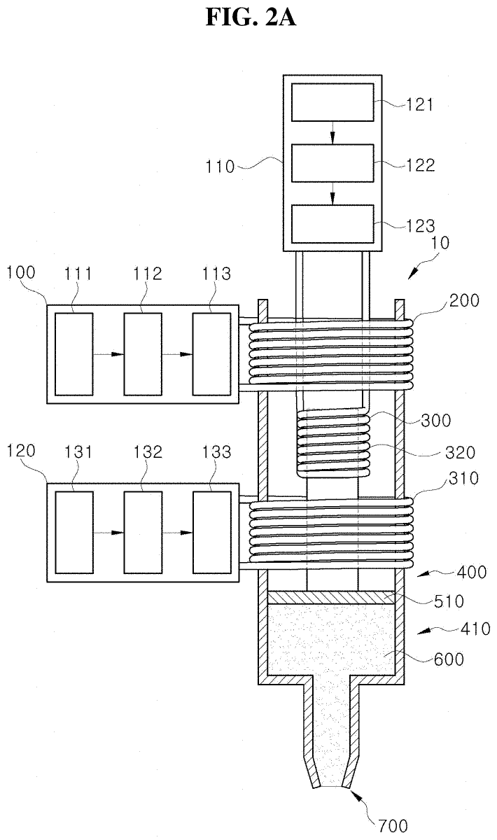

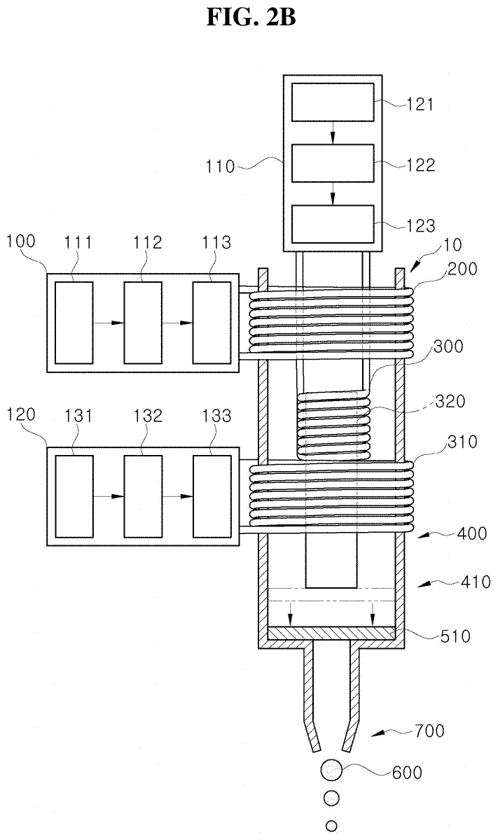

FIG. 2A is a cross-sectional view schematically illustrating a situation before voltage is applied to a needle-free injector by using a magnetic field, according to an embodiment of the present disclosure. FIG. 2B is a cross-sectional view schematically illustrating that a drug is injected by applying voltage to a needle-free injector by using a magnetic field, according to an embodiment of the present disclosure.

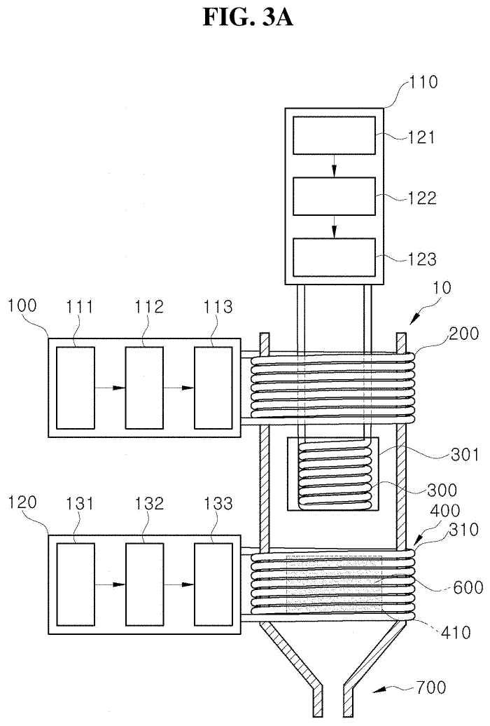

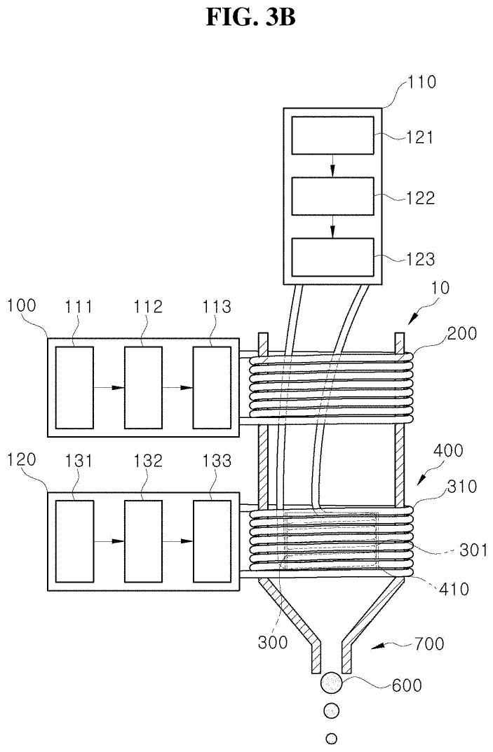

FIG. 3A is a cross-sectional view schematically illustrating a situation before voltage is applied to a needle-free injector by using a magnetic field, according to an embodiment of the present disclosure. FIG. 3B is a cross-sectional view schematically illustrating that a drug is injected by applying voltage to a needle-free injector by using a magnetic field, according to an embodiment of the present disclosure.

Referring to FIGS. 1A, 1B, 2A, 2B, 3A, and 3B, a needle-free injector 10 using a magnetic field according to an embodiment of the present disclosure includes a first current applying unit 100, a first coil 200, a second coil 300, a housing 400, a drug chamber 410, and an injection nozzle 700.

The first current applying unit 100 applies current. The first current applying unit 100 may include a positive electrode and a negative electrode. For example, the first current applying unit 100 may be a battery.

For example, the current may be a pulse current. The “pulse current” may mean current of a waveform that flows and stops at a short period. The “pulse current” may be “pulsed power”, which may increase instantaneous power by emitting a large amount of energy in a short rising time after energy is accumulated.

The rising time refers to time reaching from 10% to 90% of a pulse amplitude. In the present disclosure, the rising time may be a unit of several nanoseconds to several milliseconds. Preferably, the rising time is a unit of several nanoseconds to several microseconds.

Although not shown in drawings, the first current applying unit may further include an electric circuit that maintains the form of the generated pulse, and the electric circuit may be a pulse forming network (PFN).

The PFN may maintain the form of a pulse by preventing the form of a square pulse from collapsing due to parasitic inductance.

The needle-free injector 10 using a magnetic field may further include a switch. When the switch is turned on, current may be applied from the first current applying unit 100 to the first coil 200. When the switch is turned off, the current applied from the first current applying unit 100 to the first coil 200 may be cut off.

The first coil 200 receives the current from the first current applying unit 100. The first coil 200 may be wound with a linear material having good electrical conductivity. When the current is applied by the first current applying unit 100, a magnetic field is formed around the first coil 200.

The second coil 300 is provided between the first coil 200 and the injection nozzle 700. The second coil 300 may move to apply pressure to a drug 600. The second coil 300 may be wound with a linear material having good electrical conductivity.

The housing 400 has an enclosed accommodation space. The first coil 200 is positioned outside the housing 400. For example, the first coil 200 surrounds a part of the housing 400. The second coil 300 is positioned inside the housing 400.

For example, the housing 400 may be approximately cylindrical. A lower portion of the housing 400 may be connected to the drug chamber 410. The housing 400 may be integrated with the drug chamber 410.

The drug chamber 410 has an enclosed accommodation space. The drug chamber 410 may be provided between the second coil 300 and the injection nozzle 700.

The drug 600 is contained in the drug chamber 410. For example, the drug chamber 410 may be approximately cylindrical. A separation membrane may be disposed on the drug chamber 410. A lower portion of the drug chamber 410 may be connected to the injection nozzle 700. One side of the drug chamber 410 may be connected to a drug supply unit.

For example, the drug 600 contained in the drug chamber 410 may correspond to an amount to be injected once. When a current is applied and then the entire drug 600 contained in the drug chamber 410 is injected through the injection nozzle 700, the drug supply unit may resupply the amount to be injected once to the drug chamber 410. However, it is not limited thereto. For example, the drug 600 contained in the drug chamber 410 may correspond to an amount to be injected twice or more.

When the second coil 300 moves, pressure is applied to the inside of the drug chamber 410. Accordingly, the pressure may be applied to the drug 600. Accordingly, the drug 600 may be injected through the injection nozzle 700 and then may be injected into a user.

The injection nozzle 700 is positioned in the drug chamber 410. For example, the injection nozzle 700 may be defined as a hole at the bottom of the drug chamber 410. However, it is not limited thereto. For example, in a case in which the injection nozzle 700 is capable of injecting a drug, the injection nozzle 700 may be connected to the drug chamber 410 and may protrude from the top of the drug chamber 410 to the bottom thereof. The injection nozzle 700 injects the drug 600. The injection nozzle 700 may inject the drug 600 from the housing 400 into the drug chamber 410.

The diameter of the injection nozzle 700 may be within the range of 50 micrometers to 1000 micrometers. In a case in which the diameter of the injection nozzle 700 is less than 50 micrometers, the amount of the drug 600 injected may be small and the drug 600 may not be injected to a sufficient depth in a body of the user receiving the drug 600. In a case in which the diameter of the injection nozzle 700 is greater than 1000 micrometers, the diameter of the injected microjet increases, and thus the amount of the drug 600 that bounces off on the surface of a skin increases. Accordingly, a lot of the drug 600 may be wasted. For example, the diameter of the injection nozzle 700 may be 200 micrometers.

Although not shown in drawings, the needle-free injector 10 using a magnetic field according to an embodiment of the present disclosure may further include a drug supply unit. Although not shown in drawings, the needle-free injector 10 using a magnetic field according to an embodiment of the present disclosure may further include a drug storage unit and a check valve.

The drug supply unit receives the drug 600 from the drug storage unit and provides the drug 600 to the drug chamber 410. For example, the drug supply unit may be connected to a side surface of the drug chamber 410.

The drug storage unit may store the drug 600 provided in the drug chamber 410. The drug storage unit may be connected to the drug supply unit.

The check valve may allow the drug 600 to be delivered from the drug supply unit to only the drug chamber 410. For example, the check valve prevents the drug 600 from being delivered from the drug chamber 410 to the drug supply unit. For example, the check valve may be positioned inside the drug supply unit.

Hereinafter, a needle-free injector using a magnetic field according to an embodiment of the present disclosure shown in FIGS. 1A and 1B will be described in more detail.

As described above, the needle-free injector 10 using a magnetic field according to an embodiment of the present disclosure includes the first current applying unit 100, the first coil 200, the second coil 300, a movement block 301, the housing 400, the drug chamber 410, and the injection nozzle 700.

The first current applying unit 100 may apply current to the first coil 200. For example, the first current applying unit 100 may be a battery. For example, the voltage may be a pulse current.

The first coil 200 may be fixed. The first coil 200 may be a power coil. The power coil may refer to receiving power from the outside through a wire.

The second coil 300 is movable. When the current is applied to the first coil 200, an induction current may be generated in the second coil 300. The second coil 300 may not be connected to the outside by wiring. The “outside” may mean the outside of the housing 400. The second coil 300 may internally form a closed loop.

The second coil 300 may be a propulsion coil. The propulsion coil may refer to a coil that is propelled by being supplied only with the power induced by the power coil.

The second coil 300 may be positioned within the movement block 301. When repulsive force or attractive force acts between the first coil 200 and the second coil 300, the movement block 301 may move together with the second coil 300.

The needle-free injector 10 using a magnetic field according to an embodiment of the present disclosure may further include a locking projection 500. The locking projection 500 may control the second coil 300 such that the second coil 300 does not advance further when moving from the first coil 200 to the injection nozzle 700.

The locking projection 500 may be narrower than the housing 400. The locking projection 500 may be integrated with the housing 400. The locking projection 500 may be integrated with the drug chamber 410.

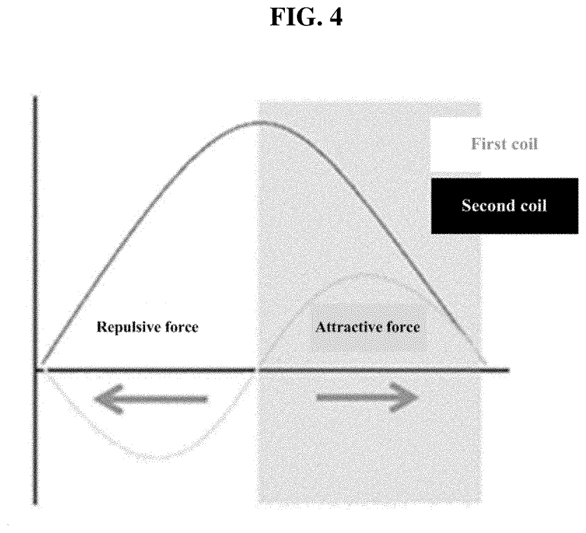

Hereinafter, referring to FIG. 4, a drug injecting method of a needle-free injector using a magnetic field according to an embodiment of the present disclosure shown in FIGS. 1A and 1B will be described.

FIG. 4 shows a current of each of a first coil and a second coil over time when a pulse current of one cycle is applied to a needle-free injector using a magnetic field shown in FIGS. 1A and 1B, according to an embodiment of the present disclosure. In FIG. 4, X axis may denote time, and Y axis may denote current.

Referring to FIGS. 1A, 1B, and 4, a pulse current of one cycle may be applied from the first current applying unit 100 to the first coil 200. When a pulse current of one cycle is applied by the first current applying unit 100, the current of the first coil 200 rises, and the magnetic field also rises. Accordingly, an induction current and an induction magnetic field are generated in the second coil 300. During the front half of one cycle, repulsive force is generated between the first coil 200 and the second coil 300 according to Faraday's law. Accordingly, the second coil 300 and the movement block 301 may move from the first coil 200 to the injection nozzle 700 such that the drug 600 is injected. At this time, the movement of the second coil 300 and the movement block 301 may be restricted by the locking projection 500.

The moving speed of the second coil 300 may be adjusted depending on the intensity of the current provided to the first coil 200. Accordingly, the speed at which the drug 600 is injected may be adjusted.

When a distance from the first coil 200 increases as the second coil 300 moves, the intensity of the induction current and induction magnetic field formed in the second coil 300 decreases.

As the current of the pulse decreases, attractive force acts on the second coil 300 and the movement block 301, which are blocked by the locking projection 500. Accordingly, during the back half of one cycle, attractive force may occur between the first coil 200 and the second coil 300 according to Faraday's law, and the second coil 300 and the movement block 301 may move from the injection nozzle 700 to the first coil 200. As the second coil 300 and the movement block 301 move from the injection nozzle 700 to the first coil 200, a drug for the next injection may be additionally supplied to the drug chamber 410.

The needle-free injector using a magnetic field according to an embodiment of the present disclosure shown in FIGS. 1A and 1B may realize both drug injection and drug provision for the next injection by providing a pulse current of one cycle.

Hereinafter, a needle-free injector using a magnetic field according to an embodiment of the present disclosure shown in FIGS. 2A and 2B will be described in more detail.

As described above, the needle-free injector 10 using a magnetic field according to an embodiment of the present disclosure includes the first current applying unit 100, the first coil 200, the second coil 300, the housing 400, the drug chamber 410, and the injection nozzle 700.

Referring to FIGS. 2A and 2B, the needle-free injector 10 using a magnetic field according to an embodiment of the present disclosure may further include a second current applying unit 110, a third current applying unit 120, a third coil 310, a first piston unit 320, and a second piston unit 510.

The second current applying unit 110 and the third current applying unit 120 apply current. In this case, for example, the current may be a pulse current. The “pulse current” may mean a current of a waveform that flows and stops at a short period. The “pulse current” may be “pulsed power”, which may increase instantaneous power by emitting a large amount of energy in a short rising time after energy is accumulated.

The rising time refers to time reaching from 10% to 90% of a pulse amplitude. In the present disclosure, the rising time may be a unit of several nanoseconds to several milliseconds. Preferably, the rising time is a unit of several nanoseconds to several microseconds.

Although not shown in drawings, the second and third current applying units may further include an electric circuit that maintains the form of the generated pulse, and the electric circuit may be a pulse forming network (PFN).

The PFN may maintain the form of a pulse by preventing the form of a square pulse from collapsing due to parasitic inductance.

A first capacitor charger 111 may charge a first capacitor 112 to provide a first current.

As the first capacitor charger 111 converts AC voltage into DC voltage and provides current to the first capacitor 112, the first capacitor charger 111 may charge the first capacitor 112.

The first capacitor 112 may store charges to provide a first current.

The first switch 113 may apply or block the first current to or from the first coil 200. For example, the first switch 113 may adjust the rising time of the first current pulse by a user. For example, the rising time of the first current pulse may be adjusted by the user from several nanoseconds to several milliseconds. It is preferably controlled from several nanoseconds to several microseconds.

The first current applying unit 100 includes the first capacitor charger 111, the first capacitor 112, and the first switch 113.

The first capacitor charger 111 may charge the first capacitor 112 to provide a first current.

As the first capacitor charger 111 converts AC voltage into DC voltage and provides current to the first capacitor 112, the first capacitor charger 111 may charge the first capacitor 112.

The first capacitor 112 may store charges to provide a first current.

The first switch 113 may apply or block the first current to or from the first coil 200. For example, the first switch 113 may adjust the width of the pulse of the first current by the user. For example, a period of the first current may be adjusted from several seconds to several milliseconds by the user.

The first switch 113 may be a solid state switch.

When the first switch 113 is turned on, the first current may be applied to the first coil 200. The first coil 200 may be a suction coil. The first coil 200 may surround a portion of the outside of the housing 400. The first current may be a pulse current.

The second current applying unit 110 includes a second capacitor charger 121, a second capacitor 122, and a second switch 123.

The second capacitor charger 121 may charge the second capacitor 122 to provide a second current.

As the second capacitor charger 121 converts AC current into DC current and provides current to the second capacitor 122, the second capacitor charger 121 may charge the second capacitor 122.

The second capacitor 122 may store charges to provide a second current.

The second switch 123 may apply or block the second current to or from the second coil 300. For example, the second switch 123 may adjust the width of the pulse of the second current by the user. For example, a period of the second current may be adjusted from several seconds to several milliseconds by the user.

The second switch 123 may be a thyristor (SCR) or a triac.

When the second switch 123 is turned on, the second current may be applied to the second coil 300. The second current may be a pulse current.

The second coil 300 may be a plunger coil. The second coil 300 may be positioned inside the housing 400. The second coil 300 may be positioned within the movement block 301. The movement block 301 may move together with the second coil 300.

The third current applying unit 120 includes a third capacitor charger 131, a third capacitor 132, and a third switch 133.

The third capacitor charger 131 may charge the third capacitor 132 to provide a third current.

As the third capacitor charger 131 converts AC current into DC current and provides current to the third capacitor 132, the third capacitor charger 131 may charge the third capacitor 132.

The third capacitor 132 may store charges to provide a third current.

The third switch 133 may apply or block the third current to or from the third coil 310. For example, the third switch 133 may adjust the width of the pulse of the third current by the user. For example, a period of the third current may be adjusted from several seconds to several milliseconds by the user.

The third switch 133 may be a solid state switch.

When the third switch 133 is turned on, the third current may be applied to the third coil 310. The third coil 310 may be an injection coil. The third coil 310 may surround a part of the outside of the housing 400. The third current may be a pulse current.

The first piston unit 320 may apply force to the second piston unit 510 to inject the drug 600. The first piston unit 320 is connected to the second piston unit 510. The first piston unit 320 and the second piston unit 510 may be connected to each other to serve as pistons.

For example, the first piston unit 320 may be made of metal, plastic, or insulating material.

At least part of the first piston unit 320 may be surrounded by the second coil 300 and the third coil 310. The first piston unit 320 may not be surrounded by the first coil 200.

The second piston unit 510 may be powered by the first piston unit 320 to inject the drug 600. The drug 600 may be injected, and the second piston unit 510 may return to its original position.

For example, the second piston unit 510 may be made of metal, plastic or insulating material.

The second piston unit 510 may be made of the same material as the first piston unit 320, or the second piston unit 510 and the first piston unit 320 may be made of different materials from each other.

In a case in which each of the first piston unit 320 and the second piston unit 510 is made of metal, the first piston unit 320 and the second piston unit 510 may affect the intensity of attractive force between the second coil 300 and the third coil 310. Accordingly, even if a relatively small current is applied, a drug may be easily injected.

Hereinafter, a drug injecting method of a needle-free injector using a magnetic field according to an embodiment of the present disclosure shown in FIGS. 2A and 2B will be described.

First of all, when the second current is applied to the second coil 300 and the third current is applied to the third coil 310, a drug is injected. In this case, the second current and the third current may be simultaneously applied. That is, the second switch 123 and the third switch 133 may be powered on at the same time. At this time, the first switch 113 may be powered off. When the second current and third current are applied respectively, the second coil 300 and the first piston unit 320 may move from the first coil 200 to the injection nozzle 700. The movement speed of the second coil 300 and the first piston unit 320 may be adjusted by adjusting the intensity of each of the second current and the third current.

The third current may be equal to or greater than the second current, but is not limited thereto. When the second current and the third current are applied, a magnetic field is formed between the second coil 300 and the third coil 310, and thus attractive force acts. Accordingly, the first piston unit 320 presses the second piston unit 510, and thus the drug 600 contained in the drug chamber 410 is injected through the injection nozzle 700.

When the drug 600 is injected, the first current may be applied to the first coil 200, and the second current may be applied to the second coil 300. The third current is not applied to the third coil 310. In this case, the first current and the second current may be simultaneously applied. That is, the first switch 113 and the second switch 123 may be powered on at the same time. At this time, the third switch 133 may be powered off. The movement speed of the second coil 300 and the first piston unit 320 may be adjusted by adjusting the intensity of each of the first current and the second current.

The first current may be equal to or greater than the second current, but is not limited thereto. When the first current and the second current are applied, a magnetic field is formed between the first coil 200 and the second coil 300, and thus attractive force acts. Accordingly, the first piston unit 320 may return to its original position, and the drug chamber 410 may be supplied with the drug 600 for the next injection.

Hereinafter, a needle-free injector using a magnetic field according to an embodiment of the present disclosure shown in FIGS. 3A and 3B will be described in more detail.

Referring to FIGS. 3A and 3B, the needle-free injector 10 using a magnetic field according to an embodiment of the present disclosure may further include the second current applying unit 110, the third current applying unit 120, the third coil 310, and the movement block 301.

The first current applying unit 100 includes the first capacitor charger 111, the first capacitor 112, and the first switch 113.

A first capacitor charger 111 may charge a first capacitor 112 to provide a first current.

As the first capacitor charger 111 converts AC current into DC current and provides current to the first capacitor 112, the first capacitor charger 111 may charge the first capacitor 112.

The first capacitor 112 may store charges to provide a first current.

The first switch 113 may apply or block the first current to or from the first coil 200. For example, the first switch 113 may adjust the rising time of the first current by a user. For example, the rising time of the first current may be adjusted by the user from several nanoseconds to several milliseconds.

The first switch 113 may be a solid state switch.

When the first switch 113 is turned on, the first current may be applied to the first coil 200. The first coil 200 may be a suction coil. The first coil 200 may surround a portion of the outside of the housing 400. The first current may be a pulse current.

The second current applying unit 110 includes a second capacitor charger 121, a second capacitor 122, and a second switch 123.

The second capacitor charger 121 may charge the second capacitor 122 to provide a second current.

As the second capacitor charger 121 converts AC current into DC current and provides current to the second capacitor 122, the second capacitor charger 121 may charge the second capacitor 122.

The second capacitor 122 may store charges to provide a second current.

The second switch 123 may apply or block the second current to or from the second coil 300. For example, the second switch 123 may adjust the width of the pulse of the second current by the user. For example, the rising time of the second current may be adjusted by the user from several nanoseconds to several milliseconds.

The second switch 123 may be a thyristor (SCR) or a triac.

When the second switch 123 is turned on, the second current may be applied to the second coil 300. The second coil 300 may be a plunger coil. The second coil 300 may be positioned inside the housing 400. The second current may be a pulse current.

The third current applying unit 120 includes a third capacitor charger 131, a third capacitor 132, and a third switch 133.

The third capacitor charger 131 may charge the third capacitor 132 to provide a third current.

As the third capacitor charger 131 converts AC current into DC current and provides current to the third capacitor 132, the third capacitor charger 131 may charge the third capacitor 132.

The third capacitor 132 may store charges to provide a third current.

The third switch 133 may apply or block the third current to or from the third coil 310. For example, the third switch 133 may adjust the rising time of the third current pulse by a user. For example, the rising time of the third current pulse may be adjusted by the user from several nanoseconds to several milliseconds.

The third switch 133 may be a solid state switch.

When the third switch 133 is turned on, the third current may be applied to the third coil 310. The third coil 310 may be an injection coil. The third coil 310 may surround a portion of the outside of the housing 400. The third current may be a pulse current.

The drug 600 may be pressed by the second coil 300 and the movement block 301 and then may be injected. The drug 600 may be injected, and the second coil 300 and the movement block 301 may return to their original positions.

Hereinafter, a drug injecting method of a needle-free injector using a magnetic field according to an embodiment of the present disclosure shown in FIGS. 3A and 3B will be described.

First of all, when the second current is applied to the second coil 300 and the third current is applied to the third coil 310, a drug is injected. In this case, the second current and the third current may be simultaneously applied. That is, the second switch 123 and the third switch 133 may be powered on at the same time. At this time, the first switch 113 may be powered off. When the second current and third current are applied, the second coil 300 and the movement block 301 may move from the first coil 200 to the injection nozzle 700. The movement speed of the second coil 300 and the movement block 301 may be adjusted by adjusting the intensity of each of the second current and the third current.

The third current may be equal to or greater than the second current, but is not limited thereto. When the second current and the third current are applied, a magnetic field is formed between the second coil 300 and the third coil 310, and thus attractive force acts. Accordingly, the second coil 300 and the movement block 301 may move to overlap the third coil 310. The second coil 300 may surround the movement block 301, and the third coil 310 may surround the second coil 300 and the movement block 301. Accordingly, the movement block 301 presses the drug chamber 410, and thus the drug 600 in the drug chamber 410 is injected through the injection nozzle 700.

When the drug 600 is injected, the first current may be applied to the first coil 200, and the second current may be applied to the second coil 300. The third current is not applied to the third coil 310. In this case, the first current and the second current may be simultaneously applied. That is, the first switch 113 and the second switch 123 may be powered on at the same time. At this time, the third switch 133 may be powered off. At this time, the second coil 300 and the movement block 301 may move slowly, and then may move faster as getting closer to the first coil 200. The movement speed of the second coil 300 and the movement block 301 may be adjusted by adjusting the intensity of each of the first current and the second current.

The first current may be equal to or greater than the second current, but is not limited thereto. When the first current and the second current are applied, a magnetic field is formed between the first coil 200 and the second coil 300, and thus attractive force acts. Accordingly, the movement block 301 may return to its original position, and the drug chamber 410 may be supplied with the drug 600 for the next injection.

The needle-free injector 10 using a magnetic field according to an embodiment of the present disclosure may have a simple structure and be easy to be miniaturized since using at least two coils.

The needle-free injector 10 using a magnetic field according to an embodiment of the present disclosure does not require a large device structure and expensive equipment costs since using at least two coils.

The needle-free injector 10 using a magnetic field according to an embodiment of the present disclosure may apply current, may move a coil, may inject a drug, and may restore a form before injection. A current may be applied once or multiple times.

Although an embodiment of the present disclosure are described with reference to the accompanying drawings, it will be understood by those skilled in the art to which the present disclosure pertains that the present disclosure may be carried out in other detailed forms without changing the scope and spirit or the essential features of the present disclosure. Therefore, the embodiments described above are provided by way of example in all aspects, and should be construed not to be restrictive.

According to an embodiment of the present disclosure, it is possible to provide a needle-free injector using a magnetic field, which has a simple structure and is easy to be miniaturized since using a magnetic field.

According to an embodiment of the present disclosure, it is possible to provide a needle-free injector using a magnetic field, which applies current, moves the coil, injects a drug, and restores a form before injection.

While the present disclosure has been described with reference to embodiments, it will be apparent to those skilled in the art that various changes and modifications may be made without departing from the spirit and scope of the present disclosure. Therefore, it should be understood that the above embodiments are not limiting, but illustrative.

What is claimed is:

1. A needle-free injector comprising:
a first current applying unit configured to apply a first current;
a first coil configured to receive the first current from the first current applying unit;
a second coil moving to apply pressure to a drug;
a housing of which the outside is surrounded by the first coil and in which the second coil is positioned;
a drug chamber which is connected to the housing and in which the drug is positioned; and
an injection nozzle positioned in the drug chamber to inject the drug.

2. The needle-free injector of claim 1, wherein the first current applying unit applies a pulse current.

3. The needle-free injector of claim 1, wherein, when the first current is applied by the first current applying unit, a magnetic field is formed in the first coil, and an induction current and an induction magnetic field are formed in the second coil,
wherein repulsive force is generated between the first coil and the second coil, and
wherein the second coil is moved by the repulsive force to apply pressure to the drug, so that the drug is injected.

4. The needle-free injector of claim 1, further comprising:
a locking projection configured to control the second coil moving when the second coil is moved.

5. The needle-free injector of claim 3, wherein the first current is a pulse current of one cycle,
wherein, during the front half of the one cycle, the repulsive force is generated between the first coil and the second coil to inject the drug, and
wherein, during the back half of the one cycle, attractive force is generated between the first coil and the second coil, and the second coil returns to its original position.

6. The needle-free injector of claim 1, further comprising:
a second current applying unit configured to apply a second current to the second coil;
a third current applying unit configured to apply a third current; and
a third coil configured to receive the third current.

7. The needle-free injector of claim 6, further comprising:
a first piston unit which is surrounded by the second coil and the third coil but is not surrounded by the first coil; and
a second piston unit connected to the first piston unit to apply pressure to the drug.

8. The needle-free injector of claim 7, wherein, when the first current is not applied to the first coil, the second current is applied to the second coil, and the third current is applied to the third coil, the second coil and the first piston unit move from the first coil to the third coil to apply pressure to the drug, so the drug is injected.

9. The needle-free injector of claim 6, further comprising:
a movement block in which the second coil is positioned.

10. The needle-free injector of claim 6, wherein, when the first current is applied to the first coil, the second current is applied to the second coil, and the third current is not applied to the third coil, the second coil moves from the third coil to the first coil to be positioned at its original position before the drug is injected.
